(19) United States
(12) Patent Application Publication
(10) Pub. No.: US 2007/0009467 A1
(43) Pub. Date: Jan. 11, 2007

(54) Spray-on makeup to simulate the appearance of wearing hosiery

(75) Inventor: Brian Charles Keller, Antioch, CA (US)

Correspondence Address: MORRISON & FOERSTER LLP, 12531 HIGH BLUFF DRIVE, SUITE 100, SAN DIEGO, CA, 92130-2040, US

(73) Assignee: Biozone Laboratories, Inc., Pittsburg, CA (US)

(21) Appl. No.: 11/175,603

(22) Filed: Jul. 5, 2005

(51) Int. Cl. A61K 8/89

(52) U.S. Cl. 424/70.12; 424/401

(57) ABSTRACT

A fast drying, spray-on cosmetic preparation containing pigments and texturants to simulate the appearance of wearing hosiery when applied to human skin. Specific tailoring of ranges of sizes for pigment and texturant particles simulates the color and texture appearance of hosiery in a durable, removable makeup that smoothes and moisturizes the skin.

### TECHNICAL FIELD

The present invention relates to a spray applied makeup containing pigment and texturant for application to a human leg, or other body part, to simulate the appearance of wearing hosiery.

### BACKGROUND ART

Throughout recent history in many cultures women, in particular, have worn hosiery to enhance the appearance of their legs. In particular, sheer hosiery made of natural materials such as silk, or artificial polymers such as nylon, have been worn to simultaneously reveal and enhance leg appearance. Sheer hosiery was originally provided as individual stockings, but is now more generally provided as integrated stocking and brief units known as “panty hose,” even though only the stocking portions are normally displayed when being worn in public. As an alternative to stockings, some women attempt to enhance leg appearance through sun tanning, although sun tanning has attendant medical side effects such as premature aging of the skin and skin cancer. Other alternatives include the use of bronzers or similar cosmetics that are topically applied. As an example, because of the scarcity of silk and nylon materials for hosiery during the Second World War, many women applied cosmetics to their legs to simulate the appearance of hosiery. Unfortunately, although applied cosmetics may simulate the appearance of a sun tan, they fail to simulate the appearance of the texture of hosiery. Moreover the applied cosmetics may be prone to transfer to clothing, prematurely wearing off, or have other durability issues.

### SUMMARY OF THE INVENTION

An embodiment of the present invention is a textured, pigmented cosmetic preparation that is applied to skin by spraying and simulates the appearance of hosiery being worn. The pigments and texturants are milled to ranges of particle sizes that are easily suspended in the cosmetic preparation and can be sprayed through a nozzle onto skin. The spraying process disburses the suspended particles in a substantially uniform pattern to simulate a substantially uniform hosiery weave. The blending of ranges of sizes for the various pigment and texturant particles simulates the color variegation and texture appearance of a fabric. The cosmetic preparation comprises a carrier solvent such as water and/or alcohol, along with emollients, humectants, viscosity enhancers, emulsifiers, and preservatives in addition to pigments and texturants. A propellant such as butane, for example, used to spray the cosmetic preparation can reduce drying time after application of the cosmetic preparation to the skin.

### BRIEF DESCRIPTION OF THE DRAWINGS

FIG. 1 displays a pressurized spray can containing a cosmetic preparation and propellant according to one embodiment of the invention.

FIG. 2 illustrates a pump spray container containing a cosmetic preparation according to another embodiment of the invention.

FIG. 3 illustrates a method of applying a cosmetic preparation according to a further embodiment of the invention.

### DETAILED DESCRIPTION OF THE INVENTION

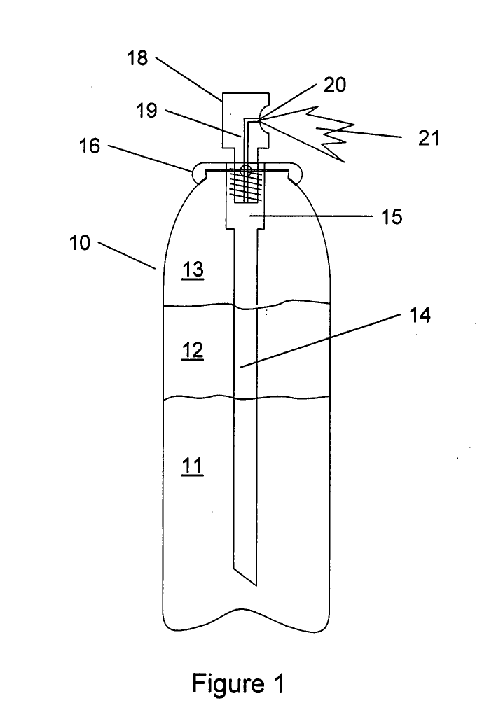

FIG. 1 illustrates a pressurized spray can 10 for dispensing a cosmetic preparation according to one embodiment of the invention. The cosmetic preparation 11 to be applied is held by gravity in the lower portion of the can 10 when the can 10 is held substantially upright. Propellant 12, (for example, butane) is present in the can with the cosmetic preparation 11. Above the cosmetic preparation 11 and propellant 12 is a gas filled region 13 that is filled with propellant vapors and possibly other gasses that forces the cosmetic preparation 11 through siphon 18, valve assembly 15, and nozzle channel 19 to spray nozzle 20 where it is emitted as a fine jet or aerosol 21 of the cosmetic preparation containing suspended pigment and texturant particles. Although the propellant 12 is illustrated as being on top of cosmetic preparation 11, propellant 12 and cosmetic preparation 11 may be at least partially miscible. Pressure seal 16 maintains the pressure of gas filled region 13. As the contents of spray can 10 are dispensed, cosmetic preparation 11 and propellant 12 levels drop, while the gas filled area 13 expands. Traditionally the spray nozzle 20 and siphon 16 assembly is outfitted with a valve assembly 15 to actuate and/or control the dispensing of the cosmetic preparation through the depression of button 18.

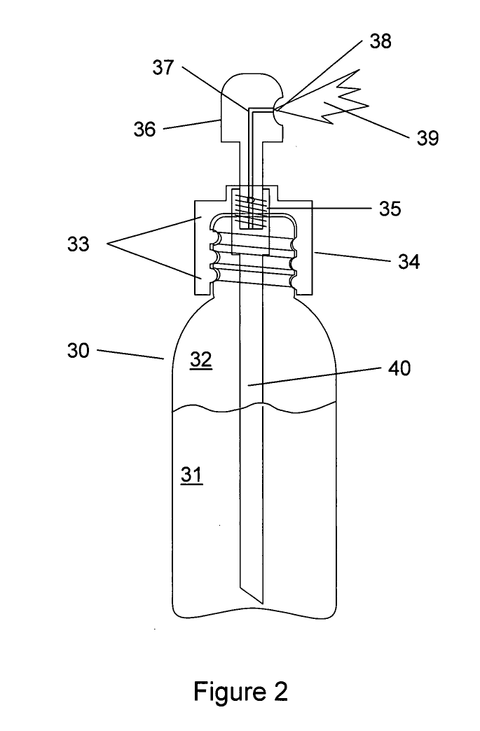

FIG. 2 illustrates a pump spray container 30 for dispensing a cosmetic preparation according to another embodiment of the invention. The cosmetic preparation 31 to be applied is held by gravity in the lower portion of the container 30 when the container 30 is held substantially upright. Container 30 has a threaded neck region 33 onto which cap 34 is screwed. Pump assembly 35 is coupled to, and penetrates cap 34 in a substantially coaxial manner, as shown. When pump assembly 35 is actuated by the depression of button 36, cosmetic preparation 31 is pumped through siphon 40, through pump assembly 35, through nozzle channel 37, and out of nozzle 38 where it is emitted as a fine jet or aerosol 39 of the cosmetic preparation containing suspended pigment and texturant particles. Region 32 of can 30 contains incidental gasses such as atmosphere and vapors of cosmetic preparation 31, at approximately one atmosphere pressure when the pump assembly 35 is not being actuated.

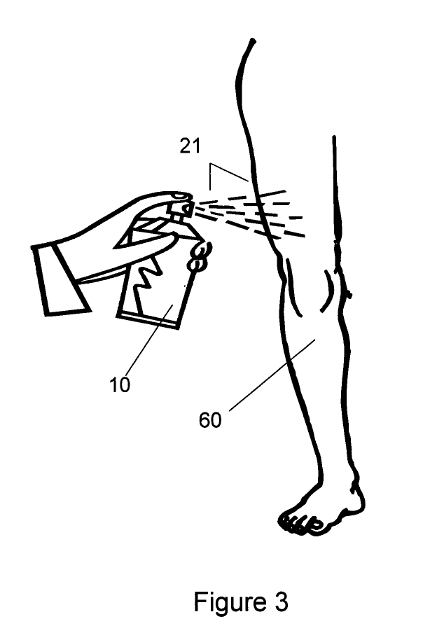

FIG. 3 depicts an example of a method of use of an embodiment of the invention. Pressurized spray can 10 is held in a substantially upright manner, actuated and moved along the surface of leg 60 in broad overlapping strokes at a typical distance of two to six inches to apply the cosmetic preparation. If complete coverage of the skin is not achieved upon first application, second and subsequent applications may be made, preferably after the prior application has dried, i.e. the carrier solvent and residual propellant have substantially evaporated. Although more time consuming than a single application, multiple applications serve to promote the evenness of coverage of the pigment and texturant.

As for the propellant used in the present invention, those widely known in the art as propellants for spray cans can be used without particular limitation. For example, in addition to butane, the following compounds may be used, without limitation: (i) liquid propane gas (LPG); (ii) chlorinated, fluorinated, and chlorinated/fluorinated low molecular weight hydrocarbons such as various Freons®; (iii) low molecular weight hydrocarbon mixtures (e.g., a mixture of butane, isobutene and propane commercially known as propellant A46 manufactured by the Phillips Chemical Co., which is an affiliate company of Phillips Petroleum Company); and (iii) ethers, and halohydrocarbons, e.g., dimethylether or dichlorodifluoromethane, or mixtures thereof with dichlorotetrafluoroethane. Mixtures of hydrocarbon and halohydrocarbon propellants with dinitrogen monoxide can be also employed. Additionally, nitrogen and carbon dioxide can also be used as propellants.

In one embodiment, the carrier solvent for the present invention is a mixture of water and ethanol, with the ethanol content being up to and including 50 percent by weight. It is well known in the field of cosmetic arts that ethanol can serve several purposes in such a solution, for example without exclusion: (i) ethanol acts as a preservative; (ii) the volatility of ethanol promotes rapid drying of the cosmetic preparation after application; and (iii) the ethanol promotes the miscibility of water with organic molecules. A disadvantage of ethanol is that it can dry out the skin when applied, necessitating the limiting of the ethanol content, and in some cases, the co-application of humectants and emollients. The ethanol may be denatured, for example by including traces of isoamyl alcohol (fusel oil) from which the ethanol may not be separated by simple distillation, as may be required by laws and regulations.

As described above, butane is used as a propellant in one embodiment of the invention. Not only can the high room temperature vapor pressure of butane provide the overpressure within the spray can to expel the cosmetic preparation, but the butane expelled as part of the cosmetic preparation quickly evaporates after application, also promoting the rapid drying of the cosmetic preparation. In various embodiments of the present invention, the content of butane in the cosmetic preparation as dispensed typically ranges from 0.0001 to about 20%, depending upon such factors as mechanical agitation of the can prior to spraying, and the content of emulsifying and organic compounds in the cosmetic preparation.

Sodium chloride (NaCl) can be dissolved in the water/ethanol mixture for a number of reasons, such as without exclusion: (i) preservation; (ii) isotonicity with perspiration; and (iii) control of the solubility of organic molecules. In various embodiments of the present invention, NaCl is added at ten percent or less by weight to prevent skin irritation and to promote miscibility and emulsification.

Cyclomethicone, dimethicone, and/or PEG/PPG-18/18 dimethicone can be mixed with the carrier solvent for inclusion as emollients in various embodiments of the invention. The concentration of one of the above, or the aggregate concentration of a combination of the above is typically 20% or less by weight. This promotes a usable viscosity for the topical application of the cosmetic preparation, as well as serving as a binding agent for the cosmetic preparation after application to the skin.

Triethylsiloxysilicate, dimethiconol, and/or decamethylcyclopentasiloxan can similarly be added, individually, or in aggregate concentrations of 20% or less by weight to contribute emollient and miscibility properties in additional embodiments of the invention.

Methyl paraben, propylparaben, butylparaben, isobutylparaben, and/or benzoyl alcohol can be included in some embodiments of the present invention as anti-microbial preservatives, either singly or in various combinations with aggregate concentrations of typically 3% or less by weight.

PEG-12 glyceryl distearate or another diacylglyceryl PEG can be included to form liposomes. PEG-12 glyceryl distearate (and some other diacylglycerol-PEG lipids in general) are known to form thermodynamically stable liposomes in aqueous solution. For example, see U.S. Pat. No. 6,610,322 to Keller et. al., herein incorporated by reference in its entirety. The liposome suspensions thus formed can be useful in delivering other compounds included in the formulation that have been encapsulated into the liposomes and to improve skin adherence and substantiveness of ingredients onto, and premeation into, the skin, improving the cosmetic appearance of the area being treated. Additionally PEG-12 glyceryl distearate or another diacylglyceryl PEG can be included in some embodiments at concentrations 10% or less by weight as a moisturizer or humefactant.

It is well known in the conventional field of foundation cosmetics that there are respective purposes for containing compounds such as describe above in connection with various embodiments of the present invention. Such purposes include, for example, moisturizing agent, humectant, non-volatile oil, emulsifier, preservative, and/or viscosity enhancer. Some of the above components can be removed from the composition, likewise additional components may be added without substantively affecting the function of the invent, as readily apparent to one of ordinary skill in the art.

In one embodiment, pigments of inorganic compound s such as iron oxides are added to provide brownish or reddish colors. These pigment particles can be milled to a variety of particle sizes ranging from nanometers to tens of microns or more, and blended to create a color with a dimensionally textured appearance rather than the appearance of a simple die. Other pigments such as titanium dioxide, zirconium dioxide, cerium dioxide, zinc oxide, chromium oxide, ferric blue, chromium hydrate, carbon black, aminosilicate polysulfide (ultramarine), manganese pyrophosphate, and certain types of metallic powder such as silver or aluminum may also be used.

The precise formulation of pigment types and particle sizes can easily be adjusted by one of ordinary skill in the art to provide a wide range of proprietary color/texture appearances, each of which can be sold as a specific, identified formulation such as shades of lipstick and other color cosmetics are presently sold. For example, some embodiments of the invention can use various proportions of iron oxide particles milled to and/or sieve separated to the approximate size classes as shown in Table 1

TABLE 1

Particle size
in Nanometers

| Size Class | Minimum | Maximum |
|---|---|---|
| I | 50 | 100 |
| II | 500 | 5000 |
| III | 10,000 | 50,000 |
| IV | 50,000 | 100,000 |

Other embodiments of the invention can use various proportions of titanium dioxide particles milled to and/or sieve separated to, for example, the approximate size classes as shown in Table 2.

TABLE 2

Particle size
in Nanometers

| Size Class | Minimum | Maximum |
|---|---|---|
| I | 50 | 200 |
| II | 3,500 | 4,500 |
| III | 10,000 | 50,000 |
| IV | 100,000 | 200,000 |

In a further embodiments, texturant particles such as mica flecks, milled to various sizes, can be included to add mechanical texture and iridescent or opalescent properties. The mica flecks can include both natural and coated mica. Mica may be coated, for example without exclusion, with titanium dioxide, iron oxide, bismuth oxychloride, and titanium. Other types of particles that may be added, without exclusion, for the purposes of texture and optical appearance include talc, silica, kaolin, Nylon® powder, polyethylene powder, Teflon®, vegetable starch, boron nitride, polymer microspheres, silicone microbeads, calcium carbonate, or magnesium carbonate hydrate.

For example, some embodiments of the invention can use various proportions of texturant particles milled to and/or sieve separated to the approximate size classes as shown in Table 3.

TABLE 3

Particle size
in Microns

| Size Class | Minimum | Maximum |
|---|---|---|
| I | 5 | 20 |
| II | 20 | 50 |
| III | 50 | 100 |

In still further embodiments the cosmetic preparation may include water or lipid soluble dyes for background coloration of the skin, that are readily known to one of ordinary skill in the art.

Table 4 summarizes ingredients and respective concentration ranges for a class of preferred embodiments for the formulation of the cosmetic preparation of the invention for dispensing from a pressurized spray can.

TABLE 4

| Ingredient | Concentration by weight (%) |
|---|---|
| ethanol | <50 |
| butane | <20 |
| cyclomethicone | <20 |
| dimethicone | <20 |
| PEG/PPG-18/18 dimethicone | <19 |
| sodium chloride | <10 |
| triethylsiloxysilicate | <12 |
| dimethiconol | <18 |
| decamethylcyclopentasiloxane | <15 |
| phenoxyethanol | <5 |
| methylparaben | <3 |
| propylparaben | <3 |
| butylparaben | <3 |
| isobutylparaben | <2 |
| benzoyl alcohol | <3 |
| PEG-12 Glyceryl Distearate | <10 (optional) |
| iron oxide | <3 |
| titanium dioxide | <5 |
| mica | <3 |
| water | (remainder of weight) |

Table 5 summarizes ingredients and respective concentration ranges for a class of other preferred embodiments for the formulation of the cosmetic preparation of the invention for dispensing from a pressurized spray can.

TABLE 5

| Ingredient | Concentration by weight (%) |
|---|---|
| ethanol | 40-50 |
| butane | 10-20 |
| cyclomethicone | 5-15 |
| dimethicone | 5-15 |
| PEG/PPG-18/18 dimethicone | 5-15 |
| sodium chloride | 2-5 |
| triethylsiloxysilicate | 5-10 |
| dimethiconol | 5-15 |
| decamethylcyclopentasiloxane | 2-10 |
| phenoxyethanol | 0.5-2 |
| methylparaben | 0.1-1 |
| propylparaben | 0.1-2 |
| butylparaben | 0.1-2 |
| isobutylparaben | 0.1-1 |
| benzoyl alcohol | 1-3 |
| PEG-12 Glyceryl Distearate | 2-8 (optional) |
| iron oxide | 0.1-3 |
| titanium dioxide | 0.1-2 |
| mica | 0.1-3 |
| water | (remainder of weight) |

The embodiments of Table 5 are particularly suited to beige, light brown, and dark brown color formulations of the cosmetic preparation.

In the event that the cosmetic preparation is dispensed from a pump spray container, rather than a pressurized spray can, the butane propellant and ethanol used in the pressurized spray can formulation of the cosmetic preparation can be omitted as shown in Tables 3 and 4, below, corresponding to Tables 1 and 2, respectively.

Table 7 summarizes ingredients and respective concentration ranges for a class of preferred embodiments for the formulation of the cosmetic preparation of the present invention for dispensing from a pump spray container.

TABLE 6

| Ingredient | Concentration by weight (%) |
|---|---|
| cyclomethicone | <20 |
| dimethicone | <20 |
| PEG/PPG-18/18 dimethicone | <19 |
| sodium chloride | <10 |
| triethylsiloxysilicate | <12 |
| dimethiconol | <18 |
| decamethylcyclopentasiloxane | <15 |
| phenoxyethanol | <5 |
| methylparaben | <3 |
| propylparaben | <3 |
| butylparaben | <3 |
| isobutylparaben | <2 |
| benzoyl alcohol | <3 |
| PEG-12 Glyceryl Distearate | <10 (optional) |
| iron oxide | <3 |
| titanium dioxide | <5 |
| mica | <3 |
| water | (remainder of weight) |

Table 7 summarizes ingredients and respective concentration ranges for a class of other preferred embodiments of the formulation of the cosmetic preparation of the for dispensing from a pump spray container.

TABLE 7

| Ingredient | Concentration by weight (%) |
|---|---|
| cyclomethicone | 5-15 |
| dimethicone | 5-15 |
| PEG/PPG-18/18 dimethicone | 5-15 |
| sodium chloride | 2-5 |
| triethylsiloxysilicate | 5-10 |
| dimethiconol | 5-15 |
| decamethylcyclopentasiloxane | 2-10 |
| phenoxyethanol | 0.5-2 |
| methylparaben | 0.1-1 |
| propylparaben | 0.1-2 |
| butylparaben | 0.1-2 |
| isobutylparaben | 0.1-1 |
| benzoyl alcohol | 1-3 |
| PEG-12 Glyceryl Distearate | 2-8 (optional) |
| iron oxide | 0.1-3 |
| titanium dioxide | 0.1-2 |
| mica | 0.1-3 |
| water | (remainder of weight) |

Although the description above contains many specificities, these should not be construed as limiting the scope of the invention but as merely providing illustrations of some of the presently preferred embodiment of this invention. For example, the cosmetic preparation is described as being dispensed from a pressurized spray can or pump spray container, but it could also be applied using a contact applicator or sprayed using an external pressure source or electrostatic techniques. Also, the size ranges for the pigments and/or texturants can be modified from those presented above as examples, in order to achieve a desired color variegation and/or texturing effect, as is well known to one of ordinary skill in the art. Thus the scope of the invention should be determined by the appended claims and their legal equivalents, rather than by the examples given.

1. A cosmetic preparation that simulates a substantially uniform pattern of variegated color and texture appearance of a fabric when applied to human skin and dried, comprising:
(a) a carrier solvent suspending a plurality of pigment particles of a plurality of pre-designated size ranges, and suspending a plurality of texturant particles of a plurality of pre-designated size ranges, such that the pigment particles and texturant particles form a substantially uniform pattern that simulates the variegated color and texture appearance of the fabric when applied to human skin and dried; and
(b) a binding agent for binding the cosmetic preparation after application.

2. A cosmetic preparation to simulate the appearance of hosiery on human skin, comprising:
(a) an aqueous solution of ethanol at fifty percent or lower concentration by weight;
(b) butane at 20 percent or lower concentration by weight;
(c) sodium chloride at ten percent or lower concentration by weight;
(d) at least one of cyclomethicone at 20 percent or lower concentration by weight, dimethicone at 20 percent or lower concentration by weight, or PEG/PPG-18/18 dimethicone at nineteen percent or lower by weight;
(e) at least one of triethylsiloxisilicate at twelve percent or lower concentration by weight, dimethiconol at eighteen percent or lower concentration by weight, or decamethylcyclopentasiloxane at fifteen percent or lower concentration by weight;
(f) at least one of phenoxyenthanol at five percent or lower concentration by weight, methylparaben at three percent or lower concentration by weight, propylparaben at three percent or lower concentration by weight, butylparaben at three percent or lower concentration by weight, isobutylparaben at two percent or lower concentration by weight, or benzoyl alcohol at three percent or lower concentration by weight;
(g) a pigment particle mixture comprising a mixture of titanium dioxide at five percent or lower concentration by weight, and an oxide of iron at three percent or lower concentration by weight; and
(h) mica at three percent or lower concentration by weight as a texturant, wherein the pigment particle mixture and the texturant form a substantially uniform pattern that simulates appearance of hosiery when applied to human skin and dried.

3. The cosmetic preparation of claim 2 in a pressurized spray can having butane as a propellant.

4. The cosmetic preparation of claim 2 wherein the oxide of iron is included as particles comprising a plurality of predetermined ranges of sizes.

5. The cosmetic preparation of claim 4 further comprising a dye for background skin coloring.

6. The cosmetic preparation of claim 2 wherein the mica is included as mica flakes comprising a plurality of predetermined ranges of sizes.

7. The cosmetic preparation of claim 2, further comprising PEG-12 glyceryl distearate at ten percent or lower concentration by weight.

8. The cosmetic preparation of claim 7 in which the PEG-12 glyceryl distearate spontaneously forms thermodynamically stable liposomes.

9. The cosmetic preparation of claim 2 wherein:
(a) the aqueous solution of ethanol is inclusively between forty and fifty percent concentration by weight;
(b) the butane is inclusively between ten and twenty percent concentration by weight;
(c) the sodium chloride is inclusively between two and five percent concentration by weight;
(d) the at least one of cyclomethicone is inclusively between five and fifteen percent concentration by weight, the dimethicone is inclusively between five and fifteen percent concentration by weight, and the PEG/PPG-18/18 dimethicone is inclusively between five and fifteen percent by weight;
(e) the at least one of triethylsiloxisilicate is inclusively between five and ten percent concentration by weight, the dimethiconol is inclusively between five and fifteen percent concentration by weight, and the decamethylcyclopentasiloxane is inclusively between two and ten percent concentration by weight;
(f) the least one of phenoxyenthanol is inclusively between one half of a percent and two percent concentration by weight, the methylparaben is inclusively between one tenth of a percent to one percent concentration by weight, the propylparaben is inclusively between one tenth of a percent and two percent concentration by weight, the butylparaben is inclusively between one tenth of a percent and two percent concentration by weight, the isobutylparaben is inclusively between one tenth of a percent and one percent concentration by weight, and the benzoyl alcohol is inclusively between one and three percent concentration by weight;
(g) the at least one of the titanium dioxide is inclusively between one tenth of a percent and two percent concentration by weight, and the oxide of iron is inclusively between one tenth of a percent and one third of a percent concentration by weight; and
(h) the mica is inclusively between one tenth of a percent and three percent concentration by weight.

10. The cosmetic preparation of claim 9, further comprising PEG-12 glyceryl distearate at between two and eight percent concentration, inclusively by weight.

11. The cosmetic preparation of claim 10 in which the PEG-12 glyceryl distearate spontaneously forms thermodynamically stable liposomes.

12. The cosmetic preparation of claim 9 wherein the oxide of iron is included as particles comprising a plurality of predetermined ranges of sizes.

13. The cosmetic preparation of claim 11 further comprising a dye for background skin coloring.

14. The cosmetic preparation of claim 9 wherein the mica is included as mica flakes comprising a plurality of predetermined ranges of sizes.

15. A cosmetic preparation to simulate the appearance of hosiery on human skin, comprising:
(a) an aqueous solution of sodium chloride at ten percent or lower concentration by weight;
(b) at least one of cyclomethicone at 20 percent or lower concentration by weight, dimethicone at 20 percent or lower concentration by weight, or PEG/PPG-18/18 dimethicone at nineteen percent or lower by weight;
(c) at least one of triethylsiloxisilicate at twelve percent or lower concentration by weight, dimethiconol at eighteen percent or lower concentration by weight, or decamethylcyclopentasiloxane at fifteen percent or lower concentration by weight;
(d) at least one of phenoxyenthanol at five percent or lower concentration by weight, methylparaben at three percent or lower concentration by weight; propylparaben at three percent or lower concentration by weight; butylparaben at three percent or lower concentration by weight; isobutylparaben at two percent or lower concentration by weight, or benzoyl alcohol at three percent or lower concentration by weight;
(e) a pigment particle mixture comprising a mixture of titanium dioxide at five percent or lower concentration by weight, and an oxide of iron at three percent or lower concentration by weight; and
(f) mica at three percent or lower concentration by weight as a texturant, wherein the pigment particle mixture and the texturant form a substantially uniform pattern that simulates appearance of hosiery when applied to human skin and dried.

16. The cosmetic preparation of claim 11 in a pump spray container.

17. The cosmetic preparation of claim 11, further comprising PEG-12 glyceryl distearate at ten percent or lower concentration by weight.

18. The cosmetic preparation of claim 13 in which the PEG-12 glyceryl distearate spontaneously forms thermodynamically stable liposomes.

19. The cosmetic preparation of claim 11 wherein the oxide of iron is included as particles milled to a plurality of predetermined ranges of sizes.

20. The cosmetic preparation of claim 11 further comprising a dye for background skin coloring.

21. The cosmetic preparation of claim 9 wherein the mica is included as mica flakes comprising a plurality of predetermined ranges of sizes.

22. The cosmetic preparation of claim 11 wherein:
(a) the aqueous solution of sodium chloride is inclusively between two and five percent concentration by weight;
(b) the at least one of cyclomethicone is inclusively between five and fifteen percent concentration by weight, the dimethicone is inclusively between five and fifteen percent concentration by weight, and the PEG/PPG-18/18 dimethicone is inclusively between five and fifteen percent by weight;
(c) the at least one of triethylsiloxisilicate is inclusively between five and ten percent concentration by weight, the dimethiconol is inclusively between five and fifteen percent concentration by weight, and the decamethylcyclopentasiloxane is inclusively between two and ten percent concentration by weight;
(d) the least one of phenoxyenthanol is inclusively between one half of a percent and two percent concentration by weight, the methylparaben is inclusively between one tenth of a percent to one percent concentration by weight; the propylparaben is inclusively between one tenth of a percent and two percent concentration by weight, the butylparaben is inclusively between one tenth of a percent and two percent concentration by weight, the isobutylparaben is inclusively between one tenth of a percent and one percent concentration by weight, and the benzoyl alcohol is inclusively between one and three percent concentration by weight;
(e) the PEG-12 glyceryl distearate is inclusively between two and eight percent concentration by weight;
(f) the at least one of the titanium dioxide is inclusively between one tenth of a percent and two percent concentration by weight, and the oxide of iron is inclusively between one tenth of a percent and one third of a percent concentration by weight; and
(g) the mica is inclusively between one tenth of a percent and three percent concentration by weight.

23. The cosmetic preparation of claim 22, further comprising PEG-12 glyceryl distearate at between two and eight percent concentration, inclusively by weight.

24. The cosmetic preparation of claim 23 in which the PEG-12 glyceryl distearate spontaneously forms thermodynamically stable liposomes.

25. The cosmetic preparation of claim 22 wherein the oxide of iron is included as particles comprising a plurality of predetermined ranges of sizes.

26. The cosmetic preparation of claim 22 further comprising a dye for background skin coloring.

27. The cosmetic preparation of claim 21 wherein the mica is included as mica flakes comprising a plurality of predetermined ranges of sizes.
